(19) United States
(12) Patent Application Publication
(10) Pub. No.: US 2009/0163785 A1
(43) Pub. Date: Jun. 25, 2009

(54) VIRTUAL NON-INVASIVE BLOOD ANALYSIS DEVICE WORKSTATION AND ASSOCIATED METHODS

(76) Inventor: RILEY H. NELSON, Orlando, FL (US)

Correspondence Address: MICHAEL W. TAYLOR, P.O. BOX 3791, ORLANDO, FL, 32802-3791, US

(21) Appl. No.: 12/339,641

(22) Filed: Dec. 19, 2008

Related U.S. Application Data
(60) Provisional application No. 61/015,247, filed on Dec. 20, 2007.

(51) Int. Cl. A61B 5/1455

(52) U.S. Cl. 600/322

(57) ABSTRACT

A virtual non-invasive blood analysis device workstation includes a light source adjacent the body part of a person for illuminating a portion of a blood vessel therein. A magnification device magnifies particles of substances in the illuminated portion of the blood vessel, and an imaging device captures images of the magnified particles. A transducer device generates electromagnetic waves based on the captured images being exposed to an electromagnetic field, with the electromagnetic waves forming color bands. Each color band corresponds to a respective particle of substance within the blood vessel. A separation chamber separates at least a portion of the color bands within the electromagnetic waves. The separated color bands represent current characteristics of a selected particle of substance within the blood vessel. A processor matches the separated color band according to the selected particle of substance with at least one of the color bands in the database, and compares the current characteristics of the separated color band to the known characteristics of the at least one matched color band.

### RELATED APPLICATION

This application claims the benefit of U.S. Provisional Application Ser. No. 61/015,247 filed Dec. 20, 2007, the entire contents of which are incorporated herein by reference.

### FIELD OF THE INVENTION

The present invention relates to the field of blood sample collection and analysis, and more particularly, to a non-invasive blood analysis through an electromagnetic separation process.

### BACKGROUND OF THE INVENTION

Across the United States and around the world, blood samples are being taken from individuals for analysis. The analysis is intended to identify and quantify substances in the individual's blood. Ideally, this is done at the time the sample is taken. The assumption is that the sample will remain pure from the time it is collected at the collection site until analyzed in a laboratory, which is usually off-site.

Blood is a key organ-tissue in the individual's body, and is an indicator of current health status, which is a predictor of future health conditions, and an identifier of anomalies within the body systems. Blood testing is a crucial diagnostic tool in medicine and health care, and even in criminal investigations. The chemical state, the physical state, and the serologic state needs to be determined and/or monitored based on the need or demand.

Samples of blood for analysis are typically obtained from individuals through an invasive process. A phlebotomist, an I.V. nurse, a registered nurse or a technician, and sometimes even a physician, will be assigned the task of collecting the sample of blood for testing/analysis. Three methods used to obtain blood samples for testing/analysis are as follows: the needle-vacuum sealed test tube combination procedure, the venous cutdown procedure, and the adoption of a secondary or tertiary role in catheterization.

In the needle-vacuum sealed test tube combination procedure, the instruments used in this process of collecting the blood sample is a needle-vacuum sealed test tube combination. The process begins by identifying a site offering the best chance of locating a vein. This exploration is performed by experienced personnel palpating some place on the forearm, arm, hand, wrist or finger until a suitable vein is found. Having located the most tactilely pulsating vein site steps to prepare the site are commenced.

The site is prepared by cleaning it with alcohol swabs, and then dried with sterile gauze. Other site preparation techniques are sometimes used, such as the use of sterile foam, wherein the cleansing substances associated therewith evaporates quickly from the skin after application.

The needle is used to puncture the skin at a selected site, usually the finger, wrist, forearm or back of the hand, but for infants, it may be the ear lobe or even the sole of the foot by the heel, in order to gain access to the vein buried in the subcutaneous tissues.

To slowdown the blood flow back to the heart, through the vein, a tourniquet may be applied (tied with a rubber band) above the intended invasion site. Once the needle pierces through the skin, epidermis and dermis and into the subcutaneous tissues, and into the vein, a collecting vessel, which is usually a vacuum-sealed test tube, is attached to the needle by piercing a hole through the vacuum-sealed top of the test tube. The blood sample is allowed to flow, upon removal of the tourniquet, into the vacuum test tube. When sufficient blood is collected, the needle is removed, and a sterile swab is pressure-applied to the site. Vacuumed test tubes are used in blood specimen collecting to reduce the chances of contamination since air contains impurities.

The second of method is the venous cutdown procedure. In rare instances where the individual's veins are very deep within the subcutaneous tissues, or even when the blood pressure is very low, sometimes due to shock or some type of illness, a procedure known as venous cutdown is used to access the vein to obtain the specimen or sample of blood for testing/analysis. Specially trained medical personnel carry out this procedure.

In the venous cutdown procedures, after cleaning the site and applying the tourniquet to the arm or leg, a small latitudinal cut is made in the skin, down into the subcutaneous tissues till a vein is found. As soon as evidence of blood appears, the needle is inserted into the exposed vein, and the sample of blood is collected, which is usually in a vacuum-sealed test tube as described above. Care must now be taken to ensure the cut is properly attended to, till it heals, and infection is prevented. Many laboratories do not carry out venous cutdown procedures as a routine method of collecting blood.

The third method is the secondary or tertiary role in catheterization. The Groshong catheter, or multi-lumen type, is used for infusion of fluids, administration of antibiotics, administration of chemotherapy, and infusion of blood. This device can also be used to draw samples of blood for diagnostic testing. The multi-lumen type catheter is used for patients with multiple CV infusion needs, and for patients with limited venous access sites who needs incompatible simultaneous multiple infusions, and for CVP monitoring. This device can also be used to sample blood for diagnostic testing. The single-lumen catheter is designed for IV therapies, infusion of antibiotics used in blood transfusion, chemotherapy administration and CV pressure monitoring. This method can also provide specimens of blood for diagnostic testing.

There are several negative features to the three above described invasive methods of collecting blood samples for analytic purposes. They are time consuming, slow, can be painful to the individual whose blood is required for testing, screening and analysis. There are risks of injury to the individual whose skin and vein must be punctured or pierced with a needle, or cut, in the venous cutdown procedure, to direct the flow of blood into the vacuum-sealed test tube.

There is a risk of infection since a foreign body is being introduced into the body. The venous cutdown procedure is fraught with risks. It needs to be performed under special conditions, with specially trained personnel. Results of blood analysis may be urgently needed, in life-saving situations. In the catheterization process, some degree of risk of interruption of prescribed medicines exists, including infection and even blood loss. There are risks of the blood samples being destroyed at any point between the site where it is collected and the laboratory where it is to be analyzed. There is also risk of injury to the individual taking the sample. Personnel have been known to accidentally prick themselves with contaminated needles.

U.S. Pat. No. 5,769,076 discloses one approach for a non-invasive blood analyzer that contains a light applicator for illuminating a detection region under the skin of a living body having a blood vessel. A camera captures an image of the illuminated detection region, and an analyzer processes the captured image and analyzes at least a component of blood in the blood vessel. The light applicator and the camera are constructed to illuminate the detection region and capture the image of the detection region through a transparent plate that is adjacent the skin. A drive controller is used to control movement of the transparent plate adjacent the skin to adjust the detection region to thereby compensate for any change in position of the blood vessel. While effective, there is still a need to improve upon how blood is sampled and analyzed in a non-invasive manner.

### SUMMARY OF THE INVENTION

In view of the foregoing background, it is therefore an object of the present invention to improve non-invasive blood analysis.

This and other objects, features, and advantages in accordance with the present invention are provided by a virtual non-invasive blood analysis device workstation comprising a support platform for supporting a body part of a person, with the body part including at least one blood vessel carrying blood. A light source may be adjacent the body part for illuminating a portion of the at least one blood vessel. A magnification device may magnify particles of substances in the illuminated portion of the at least one blood vessel.

An imaging device may capture images of the magnified particles of substances in the illuminated portion of the at least one blood vessel. A transducer device may generate electromagnetic waves based on the captured images being exposed to an electromagnetic field, with the electromagnetic waves forming a plurality of color bands, and with each color band corresponding to a respective particle of substance within the at least one blood vessel.

A separation chamber may separate at least a portion of the color bands within the electromagnetic waves, where at least one of the separated color bands represents current characteristics of a selected particle of substance within the at least one blood vessel. A database of color bands may represent known characteristics of the particles of substances within the at least one blood vessel. A processor may match the at least one separated color band corresponding to the selected particle of substance with at least one of the color bands in the database, and may compare the current characteristics of the at least one separated color band to the known characteristics of the at least one matched color band.

When examining a selected color band, a value of the chemical composition associated therewith may be determined. Determination of the value may be based on the presence or absence of particles within the chemical composition. Concentration of the chemical composition is another parameter that may be used for evaluating the particles of substances within the blood.

The virtual non-invasive blood analysis device workstation may further comprise an ultrasound device for generating an ultrasound image of the particles of substances in the at least one blood vessel, and for providing the generated ultrasound image to the transducer. Similarly, the virtual non-invasive blood analysis device workstation may further comprise an x-ray device for generating an x-ray image of the particles of substances in the at least one blood vessel and for providing the generated x-ray image to the transducer.

The virtual non-invasive blood analysis device workstation may further comprise a display for displaying the captured images of the magnified particles of substances in the illuminated portion of the at least one blood vessel. The imaging device may comprise at least one of a still camera and a video camera.

An expansion chamber may be between the transducer and the separation chamber for expanding the plurality of color bands. The separation chamber may comprise a refracting device.

The virtual non-invasive blood analysis device workstation may further comprise a temperature sensor for monitoring a temperature of the body part being illuminated by the light source, and a cooling device for cooling the illuminated body part based on the monitored temperature.

At least a portion of the light source, the magnification device and the imaging device are configured as a cuff for receiving the body part. Alternatively, at least a portion of the light source, the magnification device and the imaging device are configured as a pair of spaced apart plates for receiving the body part. The support platform may be configured for reflect light from the light source onto the body part of the person.

The virtual non-invasive blood analysis device workstation may further comprise at least one of a spectrometer and a spectroscope adjacent the illuminated body part. The virtual non-invasive blood analysis device workstation may further comprise a second transducer for converting the electromagnetic waves after separation back to images, and a second display for displaying the images.

Another aspect of the invention is directed to a method for analyzing blood using a virtual non-invasive blood analysis device workstation as described above. The method may comprise supporting a body part of a person, with the body part including at least one blood vessel carrying blood. The method may further comprise illuminating a portion of the at least one blood vessel using a light source adjacent the body part, and magnifying particles of substances in the illuminated portion of the at least one blood vessel using a magnification device.

Images of the magnified particles of substances in the illuminated portion of the at least one blood vessel may be captured using an imaging device. Electromagnetic waves may be generated using a transducer device based on the captured images being exposed to an electromagnetic field, with the electromagnetic waves forming a plurality of color bands, and with each color band corresponding to a respective particle of substance within the at least one blood vessel.

The method may further comprise separating at least a portion of the color bands within the electromagnetic waves using a separation chamber, where at least one of the separated color bands represents current characteristics of a selected particle of substance within the at least one blood vessel. A database of color bands representing known characteristics of the particles of substance within the at least one blood vessel may be provided. A processor may be operated for matching the at least one separated color band corresponding to the selected particles of substance with one of the color bands in the database, and for comparing the current characteristics of the at least one separated color band to the known characteristics of the at least one matched color band.

### BRIEF DESCRIPTION OF THE DRAWINGS

FIG. 1 is a block diagram of a virtual non-invasive blood analysis device workstation in accordance with the present invention.

FIG. 2 is a front perspective view of a virtual non-invasive blood analysis device workstation in accordance with the present invention.

FIG. 3 is rear perspective view of the virtual non-invasive blood analysis device workstation shown in FIG. 1.

FIGS. 4-7 are schematic views illustrating different designs for a virtual blood analysis sensor to be used in a virtual non-invasive blood analysis device workstation in accordance with the present invention.

FIGS. 8-10 are schematic views illustrating techniques for obtaining blood composition information in a virtual non-invasive blood analysis device workstation in accordance with the present invention.

FIG. 11 is a block diagram illustrating a virtual blood analysis interphase controller for the virtual non-invasive blood analysis device workstation shown in FIG. 2.

FIG. 12 is a block diagram illustrating virtual blood analysis display information for the virtual non-invasive blood analysis device workstation shown in FIG. 2.

FIG. 13 illustrates a prism to be used in the separation chamber within the virtual non-invasive blood analysis device workstation in accordance with the present invention.

FIG. 14 is a block diagram of a high speed photographic device with a micrometer to be used within a virtual non-invasive blood analysis device workstation in accordance with the present invention.

FIG. 15 is a block diagram illustrating different phases of the virtual non-invasive blood analysis device workstation including networking in accordance with the present invention.

FIG. 16 illustrates a typical laboratory order and analysis result sheet in accordance with the present invention.

### DETAILED DESCRIPTION OF THE PREFERRED EMBODIMENTS

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

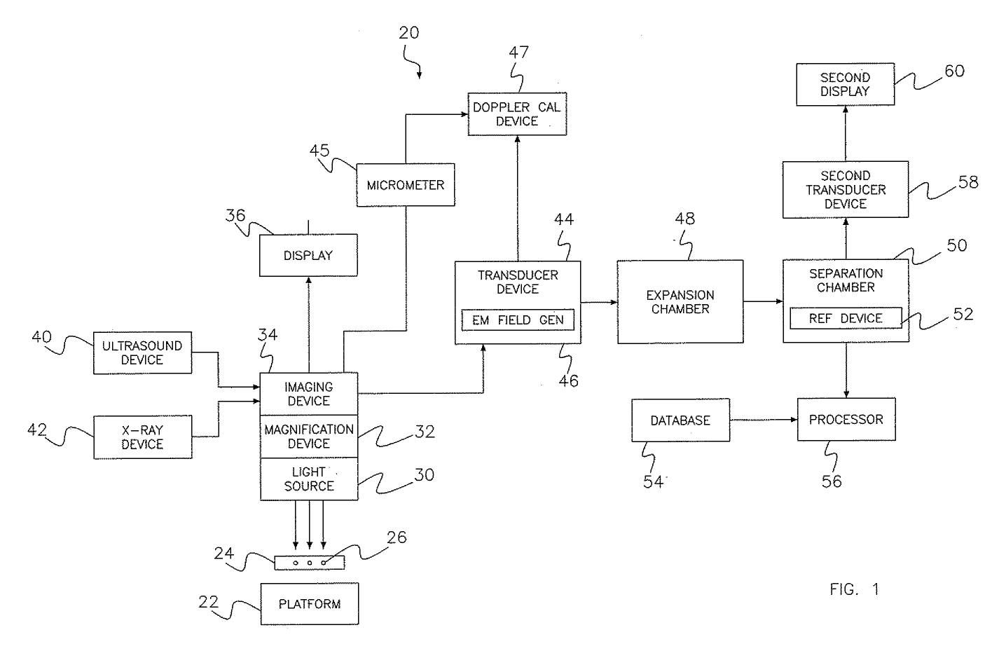

Referring initially to FIG. 1, a virtual non-invasive blood analysis device workstation 20 includes a support platform or interphase 22 for supporting a body part 24 of an individual or patient. The body part 24 includes blood vessels 26 carrying blood. A light source 30 is adjacent the body part 24 for illuminating a portion of the blood vessels 26. A magnification device 32 magnifies particles of substances in the illuminated portion of the blood vessels 26. The magnification is to the molecular/microscopic level. The support plate 22 may advantageously be configured as a refractive/reflective surface to help provide a three dimensional view of the blood vessel 26, enabling the capture of the images of particles in the blood.

As will be discussed in greater detail below, the particles of substance refer to molecules of specific chemicals within the blood. Each specific chemical has a known atomic weight. Also, the different chemicals emit different spectrum of light. In other words, they have different wavelengths or color bands associated therewith. By focusing on selected color bands within electromagnetic waves, the blood can be analyzed.

An imaging device 34 captures images of the magnified particles of substances in the illuminated portion of the blood vessels 26. The captured images may appear on a display screen 36 for viewing. An ultrasound device 40 and an x-ray device 42 may also be used to enhance or add to the captured images.

A transducer device 44 is downstream from the imaging device 34 for generating a plurality of electromagnetic waves based on the captured images being exposed to an electromagnetic field. The electromagnetic field may be generated by an electromagnetic field generator 46 within the transducer device 44. Alternatively, the electromagnetic field generator 46 may be separate from the transducer device 44.

A Doppler calculation device 47 is coupled to the transducer device 44 for determining movement of the particles within the blood vessels 26. The Doppler calculation device 47 also interfaces with a micrometer 45. The micrometer 45 is used to measure the length of the blood vessels, and provides this information to the Doppler calculation device 47 so that information on the blood can be obtained.

To assist between the static images generated by the imaging device 34 and the dynamic images generated by the transducer device 44, additional processing may be used. Similar to animated objects being manipulated, such as cartoons, so will the pictures of particles, atoms, ions or molecules, or the electromagnetic waves of the same. Combination of a high intensity light source, photographic/videographic, ultrasonography and an ultrasonograph can be used to visualize and capture images of molecules, atoms, ions and particles in the blood, for analytical purposes, in order to arrive at the desired concentration or other indices.

Electromagnetic radiation takes the form of self-propagating waves in a vacuum or in matter. Electromagnetic radiation has an electric and magnetic field component which oscillate in phase perpendicular to each other and to the direction of energy propagation. Electromagnetic radiation is classified into types according to the frequency of the wave, these types include (in order of increasing frequency): radio waves, microwaves, terahertz radiation, infrared radiation, visible light, ultraviolet radiation, X-rays and gamma rays. Of these, radio waves have the longest wavelengths and Gamma rays have the shortest. A small window of frequencies, called visible spectrum or light, is sensed by the eye of various organisms, with variations of the limits of this narrow spectrum.

In the illustrated embodiment, the electromagnetic waves form a plurality of color bands, with each color band corresponding to a respective particle of substance within the blood vessels 26. By focusing on selected color bands within electromagnetic waves, the blood can be analyzed.

An expansion chamber 48 is used for expanding the color bands. The expansion separates partially overlapping color bands, as well as enhancing the color bands at the lower and upper ends of the spectrum. The expansion may be characterized as moving away from the focal point of a light, wherein the light becomes wider in terms of viewing the further away one stands. In contrast, moving closer to the light causes the light to be viewed narrower.

A selection chamber 50 is used for selecting one or more color bands for analysis. In one embodiment, the selection chamber 50 includes a refraction device 52, such as a prism, for example, for separating out the different color bands. There are typically hundreds of color bands, and depending on the desired analysis of the blood, the appropriate color bands are selected. The selected color bands are then compared to a database 54 of color bands. Each color band has a frequency associated with it, which in turn corresponds to a particular chemical within the blood. The database 54 is created based on an analysis on real blood, and these known characteristics are compared to the virtual characteristics obtained by the workstation 20.

The separation chamber 50 thus separates at least a portion of the color bands within the electromagnetic waves, where at least one of the separated color bands represents currently known characteristics of the particle of substance within the blood vessel 26. The database 54 of color bands represent known characteristics of the particles of substances within the blood vessel 26.

A processor 56 matches the at least one separated color band with one of the color bands in the database 54, and compares the current characteristics of the at least one separated color band to the known characteristics of the matched color band. The database 54 may be in a memory separate from the processor 56, or may be included as part of the processor.

When examining a selected color band, the value of the chemical composition associated therewith may be determined. Determination of the value may be based on the presence or absence of particles within the chemical composition, as readily appreciated by those skilled in the art. Concentration of the chemical composition is another parameter that may be used for evaluating the particles of substances within the blood.

As an example, different color bands may correspond to the following: red blood cells, white blood cells, platelets, chemical substances within the blood itself, and determination of particles in the blood that should not be in the blood.

The molecules and particles of substances in the blood have different atomic weights and configurations, and therefore, are identifiable, separable, differentiable, and measurable in terms of concentration and other indices. Because of differences in atomic weights and configurations, different molecules, atoms, ions and particles will reflect electromagnetic rays/waves differently. Different molecules, atoms, ions and particles in the blood will be at different, calculable and of variable concentrations. The different molecules, atoms, ions and particles will group or cluster based on molecular weight and concentrations, by appropriate means.

The illustrated virtual non-invasive blood analysis device workstation 20 further includes a second transducer 58 for converting the electromagnetic waves after separation back to images, and a second display 60 for displaying the images.

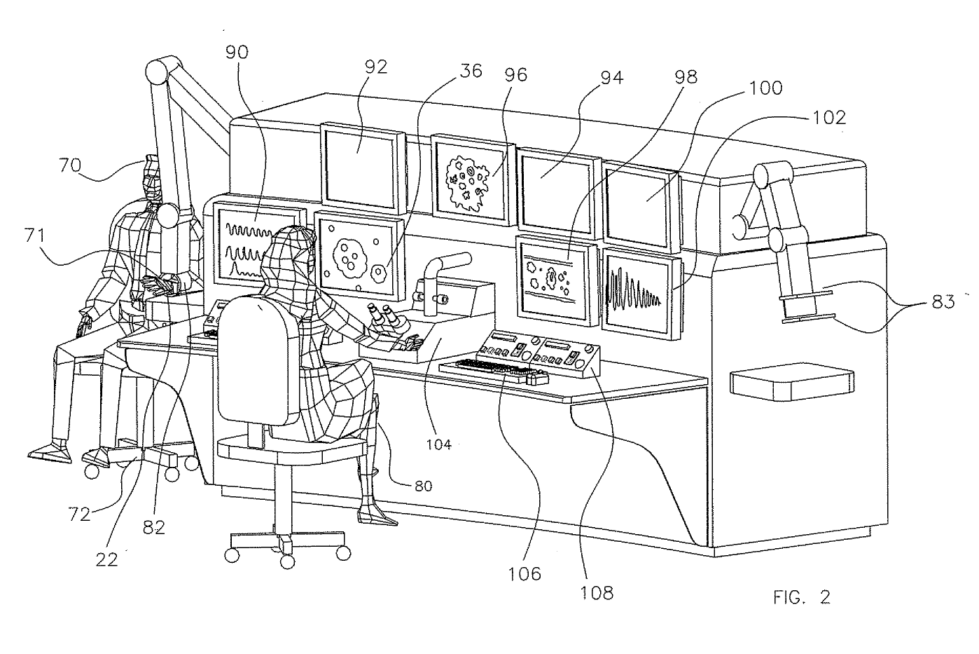

A forward perspective view of the virtual non-invasive blood analysis device workstation 20 will now be discussed in reference to FIG. 2. The individual or patient 70 sits in a chair 72 and places their arm on the support plate 22, which is also referred to as an interphase. The virtual non-invasive blood analysis device workstation 20 is controlled by an operator 80.

In the illustrated embodiment, a cuff 82 fits around the arm 71 of the individual 70. The cuff 82 interfaces with at least a portion of the light source 30, the magnification device 32 and the imaging device 34. The cuff 82 may further interface with a spectroscope and a spectrometer. On the other side of the workstation 20, is another sensor for another individual. Instead of a cuff 82, the sensor is configured as a pair of spaced apart plates 83. Alternatively, the sensor may be configured with a single plate.

The workstation 20 includes a number of displays. There is a display 90 for the wave separation, i.e., associated the separation chamber 50. There is a display 36 for the imaging device 34, which may be photography and/or video equipment. There is a main display 92 and a backup display 94 for the workstation 20. There is a display 96 for an ultra-microscope. There may also be an additional display 98 for displaying images produced from the light sensors 30 without magnification. There is a display 100 for high speed photographic equipment. The electromagnetic waves produced by the transducer device 44 are viewed on display 102. The workstation 20 further includes a magnifying device 104. Operator interface to the workstation 20 is in the form of a keyboard 106, for example, and a function selection panel 108.

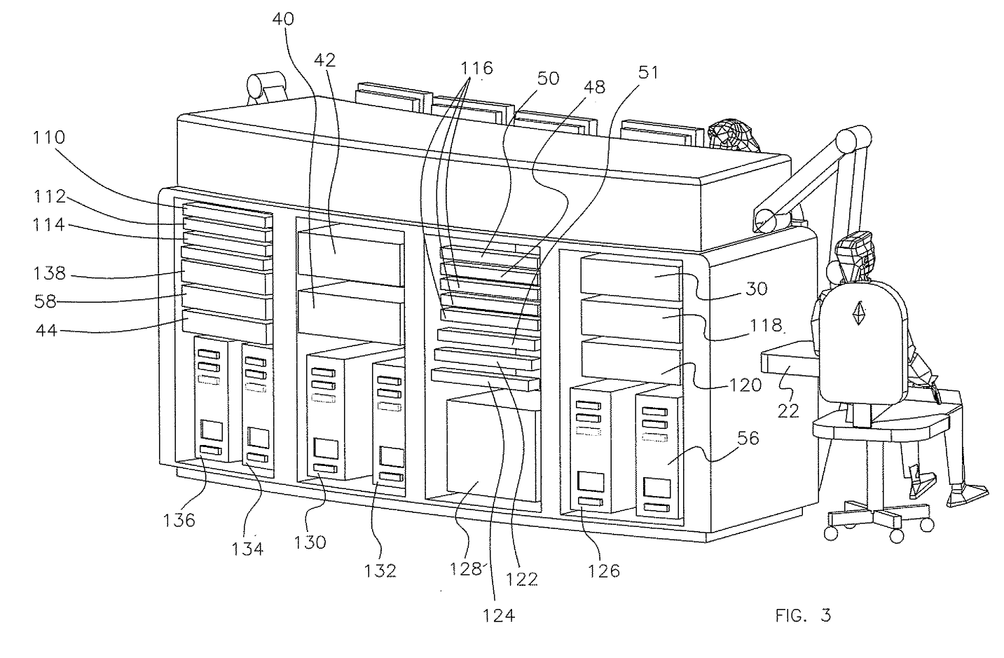

The equipment making up the virtual non-invasive blood analysis device workstation 20 will now be discussed with reference to the rear view provided in FIG. 3. The interphase or support platform is indicated by reference 22. Element 110 is an Internet hub for connecting to the Internet. Element 112 is an electromagnetic wave refractor. Element 114 is a Doppler device that cooperates with the ultrasound device 40, which is also used to provide an input to the imaging device 34. Element 114 may also cooperate with the Doppler calculation device 47 for comparing the frequencies of wavelength motion. Yet another device used to provide an input to the imaging device 34 is an x-ray device 42. Reference 50 is the separation chamber and reference 51 is a sorting chamber, and reference 48 is the expansion chamber.

The workstation 20 may include multiple databank reference chambers, as indicated by reference 116. The high intensity light source is indicated by reference 30, which provides a light to the cuff 82. A cooling device 118 may be used to provide cooling to the interphase 22 and/or to the cuff 82. Reference 120 is a high intensity magnetic device. The main processor for the workstation is indicated by reference 56.

The spectroscope is indicated by reference 122 and the spectrometer is indicated by reference 124. A standby processor for the workstation 20 is indicated by reference 126. An electrical power source for the workstation 20 is indicated by reference 128. Front end data processors are indicated by indicated by references 130 and 132. Reference 134 is a temperature monitor for monitoring the temperature of the interphase 22 and/or cuff 82.

Reference 136 is a microspectrophotometer. Reference 44 is the transducer device for changing captured images to electromagnetic waves, and reference 58 is the second transducer device for changing the electromagnetic waves back to images. Reference 138 is a high speed photographic device.

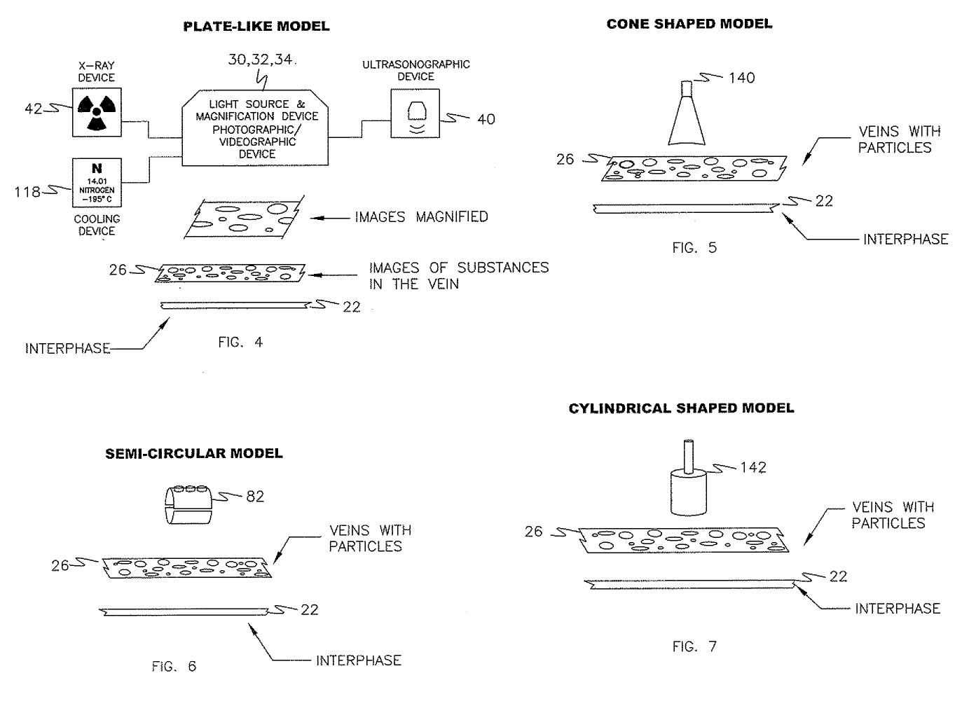

FIGS. 4-7 are schematic views illustrating different designs for a virtual blood analysis sensor to be used in the virtual non-invasive blood analysis device workstation 20. A plate-like model is provided in FIG. 4. The plate-like model corresponds to reference 83 shown in FIG. 3. The light source 30, magnification device 32 and photo/video device 34 interface with the plate like model. Inputs to the photo/video device 34 include an ultrasound device 40 and an x-ray device 42. Depending on the generated temperatures during testing, a cooling device 118 is used to protect the skin of the individual 70 being tested.

A cone-shaped model sensor 140 is provided in FIG. 5. A semi-circular model sensor 82 is provided in FIG. 6, which corresponds to the cuff shown in FIG. 2. A cylindrical shaped model sensor 142 is provided in FIG. 7.

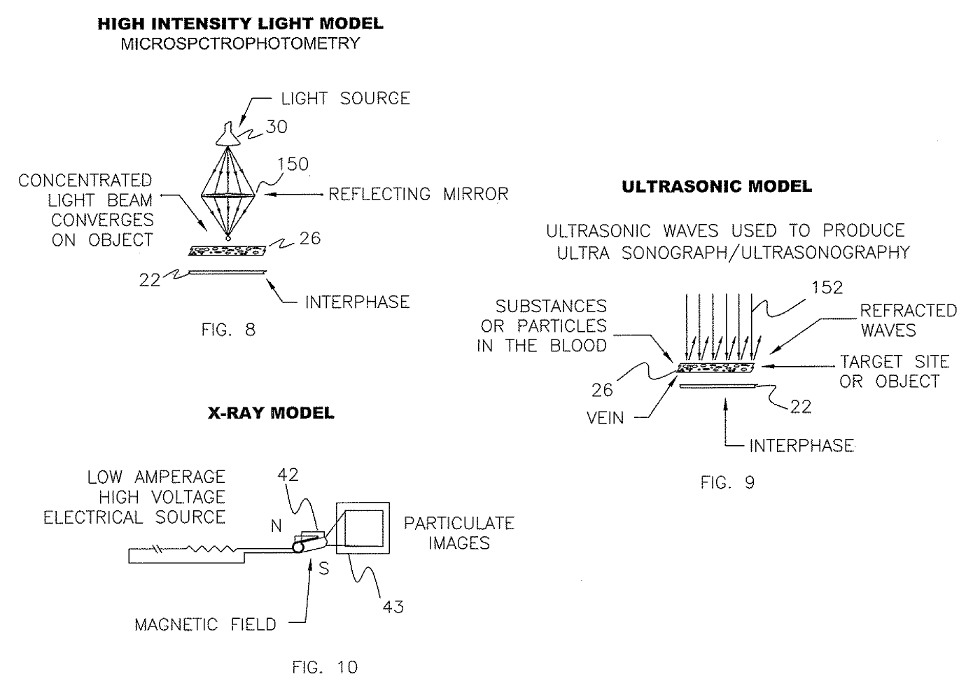

FIGS. 8-10 are schematic views illustrating techniques for obtaining blood composition information in the virtual non-invasive blood analysis device workstation 20. As illustrated in FIG. 8, a high intensity light source 30 directs light through a reflecting mirror 150 so that the light is concentrated on a blood vessel 26. As illustrated in FIG, 9, ultrasound waves 152 from an ultrasound device 40 are used to produce an ultrasonograph. The sound waves are refracted by the blood vessels 26. Yet another method of obtaining blood composition information is to use x-rays or modified x-rays 43 generated by an x-ray device 42, as illustrated in FIG. 10.

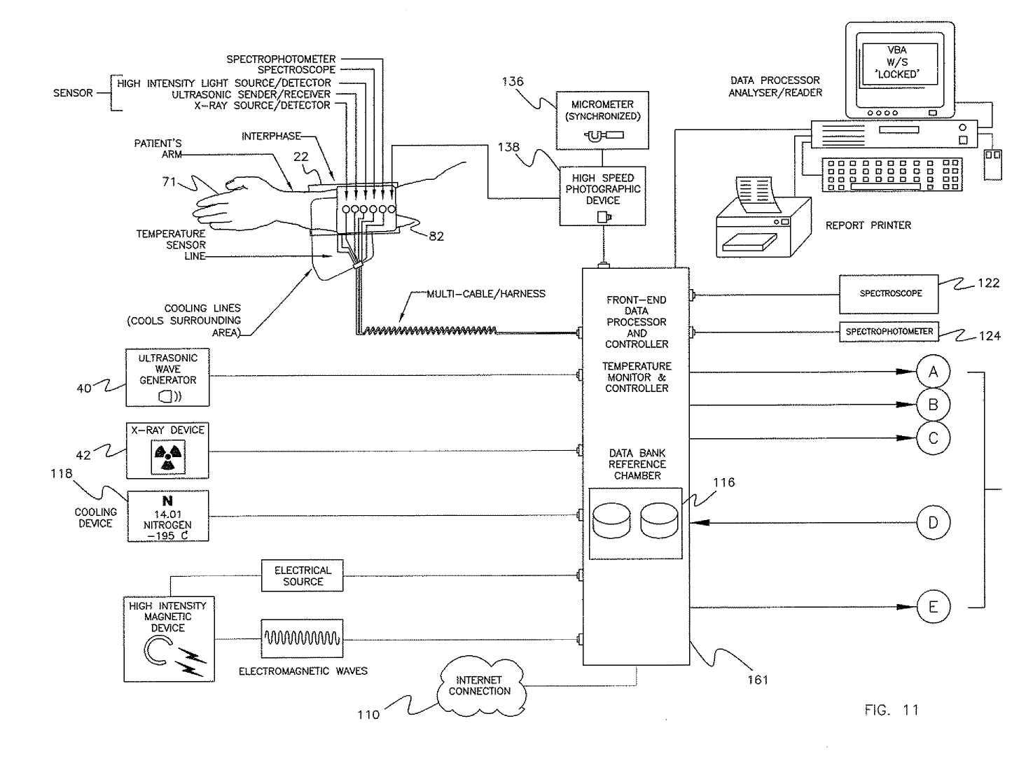

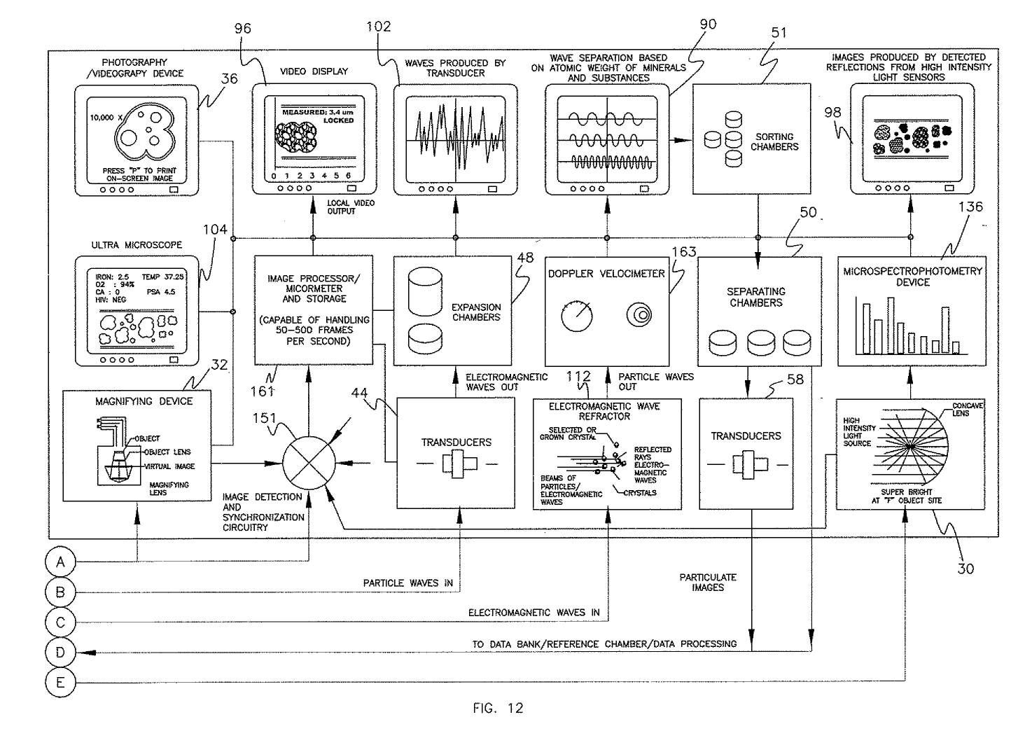

FIG. 11 is a block diagram illustrating a virtual blood analysis interphase controller 161 for the virtual non-invasive blood analysis device workstation 20. The main processor 56 for the workstation 20 controls interface to the interphase or support plate 22. The front-end data processor and controller 161 interfaces with a number of different items, including a harness coupled to the cuff 82. Connections A-E interface with the same corresponding connections A-E provided in FIG. 12. FIG. 12 is a block diagram illustrating virtual blood analysis display information for the virtual non-invasive blood analysis device workstation shown 20.

Images from the magnification device 32 are displayed on display 36. The images are provided to an image detection and synchronization circuit 151. The high intensity light source 30 provides the light for the magnification device 32. The output of the image detection and synchronization circuit 151 is provided to an image processor/micrometer and storage device 161. This circuit may be separate from the main processor 56. Alternatively, this circuit may be part of the main processor 56. The output of the image processor xx may be viewed on video display 96.

The particle waves are provided to a transducer device 44 for providing electromagnetic waves to an expansion chamber 48. The electromagnetic waves produced by the transducer device 44 may be viewed on display 102. An electromagnetic wave refractor 112 is used to help separate the electromagnetic waves. The Doppler or speed of the electromagnetic waves may be controlled and measured with the assistance of a Doppler controller 163 that interfaces with the Doppler calculation device 47.

The separated electromagnetic waves are viewed on display 90. A sorting chamber 51 is used to sort the color bands which are of interest. With the help of a sorting device, such as a prism, for example, the desired electromagnetic waves are separated in the separation chamber 50. If the separated particles corresponding to the selected color waves are to be viewed, then a second transducer device 58 convert the separated electromagnetic waves back to particles for viewing on display 60.

The high intensity light source 30 may be used to allow a microspectrophotometry device 136 to generate a graph or histogram of the color waves. The output of the microspectrophotometry device 136 is provided to a display.

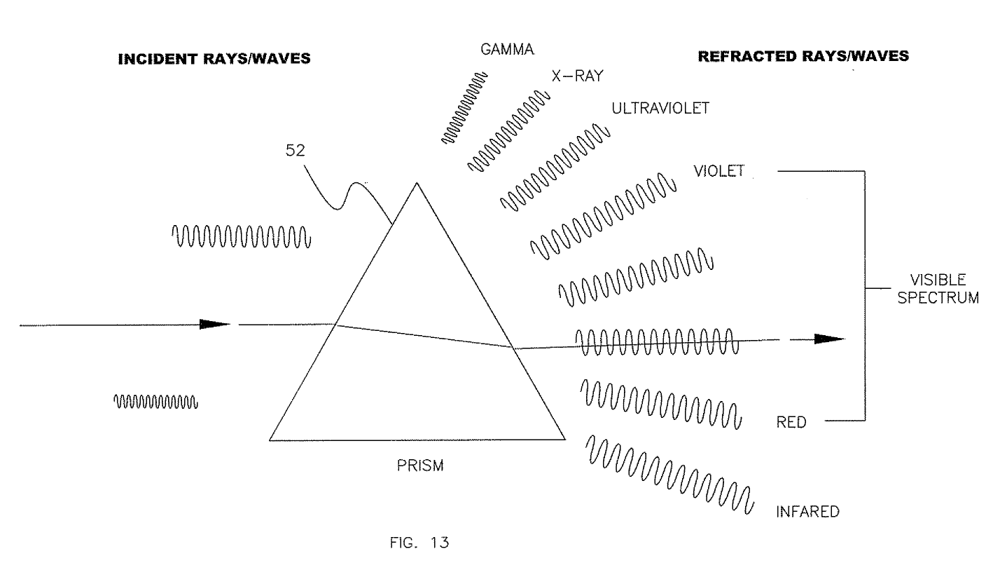

FIG. 13 illustrates a prism 52 that may be used as the refracting device for separating the electromagnetic waves. The electromagnetic waves are applied as input to the prism and are then refracted to different positions or wavelengths within the spectrum.

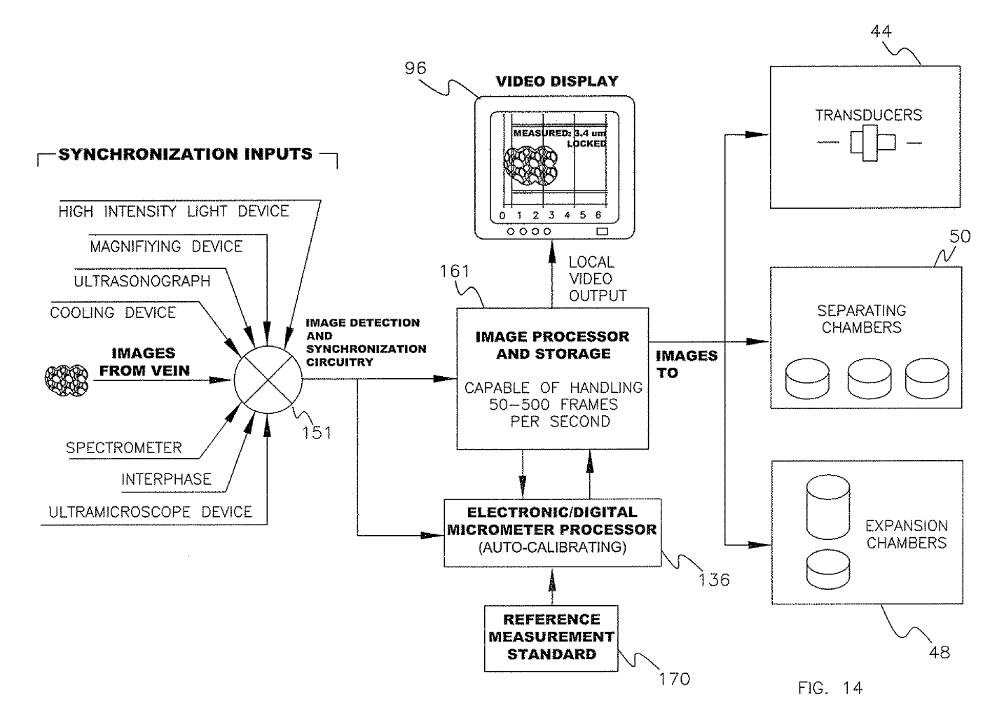

FIG. 14 is a block diagram of a high speed photographic device with a micrometer processor 136 used within the virtual non-invasive blood analysis device workstation 20. Reference measurement data 170 is stored in a memory, and is coupled to the processor 161. The micrometer processor 136 cooperates with the micrometer 45 and measures the length of the portion of the blood vessel, and compares with the speed of the electromagnetic waves. The speed of the electromagnetic waves can then be determined based on the color band spectrum.

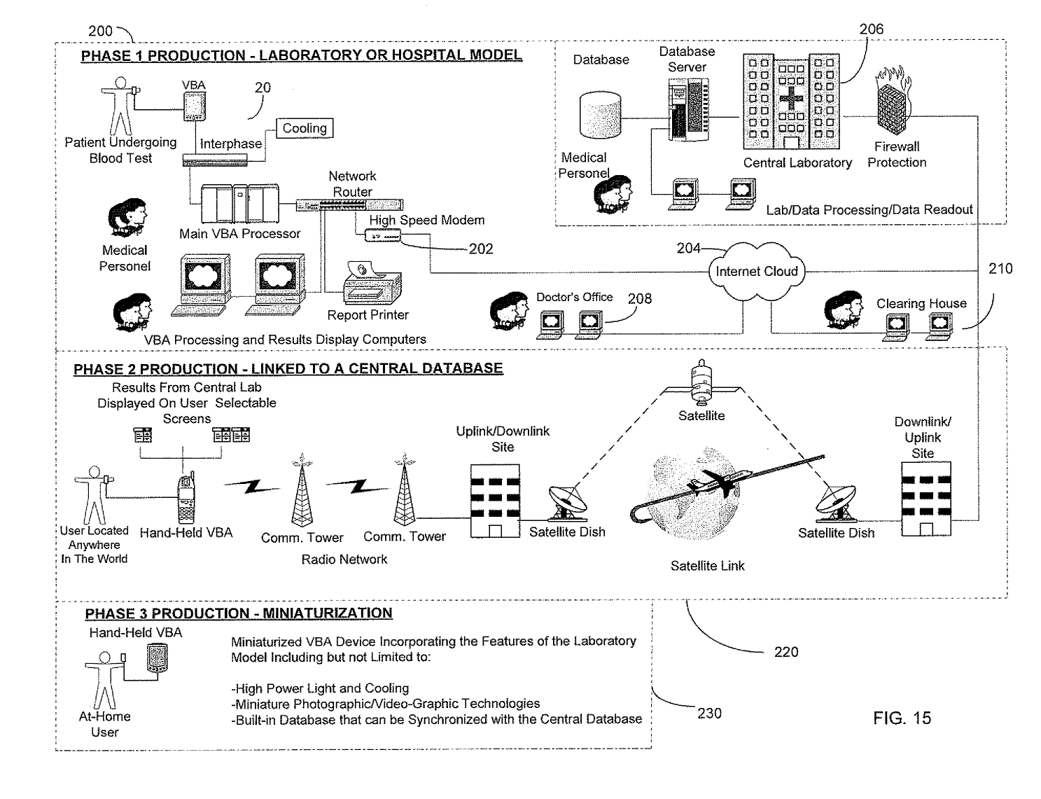

FIG. 15 is a block diagram illustrating different phases of the virtual non-invasive blood analysis device workstation 20 including networking. In phase 1 200, the virtual non-invasive blood analysis device workstation 20 interfaces with a modem 202 for communicating over the Internet 204. The results may be provided to a laboratory or hospital 206 for example. The results may also be provided to a doctor's office 208 and a clearing house 210.

In phase 2 220, the virtual non-invasive blood analysis device workstation is now configured more compactly as a hand-held device. The hand-held device includes a telephone for communicating the blood samples to a central database for determining the results. The central database may be at the laboratory or hospital 206, for example. The results of the blood analysis are then communicated back to the user. In phase 3 230, the hand-held device is self-contain for analyzing the user's blood without having to communicate to a central database.

The virtual non-invasive blood analysis device workstation 20 eliminates the need to draw blood from the patient or individual. In theory, the workstation 20 uses a well know combination of processes known in chemistry for over 90 years (i.e., mass spectrometry/mass spectrograph), along with modern technologies, to meet the needs of millions of people around the world: testing and screening procedures.

The process is fast, convenient, and pain free. As illustrated in FIG. 15, the functions of the workstation 20 are provided in a hand held device to make it even more convenient and available to the public. Just like a cellular phone is capable of fitting into the palm of the user's hand, so will this device.

Alternatively, in the interim between the full size workstation and the miniaturized palm-held version, there will be a hand held version capable of capturing the picture of the substances in the blood and transmitting this picture via satellite (just like cellular phone or road navigation systems) to a central laboratory. At the central laboratory, the necessary equipment is available to transform these incoming pictures of blood particulate content into indices, based on the requirements. Results will be transmitted back to the individual's hand held device, within minutes or as needed.

The molecules and particles of substances in the blood have different atomic weights and configurations, therefore, are identifiable, separable, differentiable, and measurable in terms of concentration and other indices. Because of differences in atomic weights and configurations, different molecules, atoms, ions and particles will reflect electromagnetic rays/waves differently.

Different molecules, atoms, ions and particles in the blood will be at different, calculable and of variable concentrations. The different molecules, atoms, ions and particles will group or cluster based on molecular weight and concentrations, by appropriate means.

Similar to animated objects being manipulated, such as cartoons, so will the pictures of particles, atoms, ions or molecules, or the electromagnetic waves of the same. Combination of a high intensity light source, photographic/videographic, ultrasonography and an ultrasonograph can be used to visualize and capture images of molecules, atoms, ions and particles in the blood, for analytical purposes, in order to arrive at the desired concentration or other indices.

Magnetic field and/or electric field, jointly or separately, will behave like a cathode; moving particles or particle-waves, or pictures of the same (like the process of animation). Images of the particles/waves can be magnified, in conjunction with the ultrasonography, so that these images or sonograms can be differentiated on a screen.

The workstation 20 can identify the presence of certain molecules and particles in the blood more easily using an electromagnetic separation process. This may be tested by using physical means with respect to fluids. For example, pour unmeasured volumes of different kinds of oil and other liquids including castor oil, corn oil, sunflower seed oil, olive oil, distilled water, vinegar, for example, into a tall graduated glass tube, closed at one end, then shake well to form a mixture. Allow the mixture to settle for a few minutes to an hour.

All the different liquids will separate out of the mixture, and settle in the tube based on the density or specific gravity (at constant temperature) of each liquid in the graduated tube. The volume of each liquid can then be measured, by examining their levels, in the graduated tube.

We can measure the concentration of the different molecules, atoms, ions and particles in the blood by the use of reference standards. The identity of substances in the blood can be done by reference standards: referencing blood collected in the conventional method and then programming blood characteristics into a database or a data bank. The programming system will provide the capability of identifying and separating each blood particle's characteristics. Again, this may be done by the use of reference standard methods. This is based on the use of information gathered from blood collected using conventional methods. Each sets of particles separated can be isolated from all other particles. By isolating and identifying each particle, the concentration and other indices can be easily measured.

As discussed above, a description of the device will be provided again. A sleeve or cuff wraps around the arm or hand of the individual. Alternatively, a flat or curved plate-like or cylindrical sensor, or a conical or semi-circular sensor may be placed in direct contact with the skin of the individual in the area selected to be scanned. The sleeve/cuff or plate may be capable of operating with certain material covering the area of contact, such as a vest or shirt.

Incorporated into are interfacing with the patient contact area sleeve (e.g., configured as a cuff, cylindrical, conical area or semicircular area) are the following:

a) a high intensity light source;

b) an ultrasound/ultrasonagraphic device to visualize and capture the images/sonograms of molecules, ions, atoms and particles in the blood, combined with, or alternative to an ultramicroscope to view tiny, sub-microscopic particles or images or sonograms of molecules, atoms, ions and particles, combined with a photography/videography device to record information produced by ultrasonography images, or a sonogram combined with a magnetic source of determinable/variable strengths to provide the magnetic field, to align the molecules, atoms, ions and particles, and the magnetic waves when created by a transducer;

c) a magnifying device of variable magnification, and with high magnification resolution;

d) a transducer to change the picture of molecules, atoms, ions, and particles to electromagnetic waves, and conduct/direct these waves to an expansion chamber; and

e) an electrical source, which when combined with the magnetic field, changes the particulate pictures into electromagnetic waves.

A cooling system (such as liquid nitrogen) to protect possible tissue damage from the high intensity light and high intensity ultrasound waves;

A microspectrophotometry device which uses the ultraviolet spectrum of the high intensity light source to provide a histochemical study; quantitative and qualitative, of the liquid portion of the blood.

A Doppler velocimeter which measures the flow of the blood and since the images/sonograms of the blood in the veins is being captured are in motion, this calculation may be factored into the calculation of the wave lengths of the different substances in the blood.

A combined spectrophotometer/spectrometer to determine the intensity of various wavelengths emitted by the different substances in the blood, and is equipped with scales to measure the wavelengths or other indexes of refraction.

Another transducer to change wavelengths back into particulate beams, if necessary.

A sorting chamber to sort out and select the item(s) in the blood sample required for testing/analysis, in conjunction with a computerized data bank including a database recorded from blood taken in the conventional way.

A display screen for allowing the index/indices to be more easily and quickly read.

The light source is strong enough to illuminate the site, down to the subcutaneous tissue, to the venous level, combined with ultrasonography technology. The magnetic force, which attracts and separates the molecules and particles, combined with, the electric current, which aids in the ionization of some particles and molecules, and the direction of motion of the ionized particles/waves. The magnifier magnifies the molecules, particles and ions, thus enabling isolation of particles or molecules or ions, as necessary.

At this point, the images/ultrasonograms/sonograms produced by ultrasound, the spectrometer, the microspectrophotometry device, and the ultramicroscope, separate or combined, and captured by the photographic/videographic devices, can be analyzed, because each substance in the blood emits different images. Nevertheless, chances are that these images will be so numerous, that sorting out one or two specific items to measure such an index as concentration of a specific blood content would be somewhat tedious, cumbersome or difficult. There are scores of items in blood, any one or more of which may be required to be analyzed and reported on, or used for medical purposes, or even criminal investigations.

Therefore, the next phase is to use the transducer to change these images to all waves, and then sort out or separate out the required/identifiable waves for analysis by the separation chamber, and computerized database/data analysis/readout can be eliminated. The transducer changes and conducts the images of the molecules and particles from graphic images to electromagnetic waves, sending them to the sorter. The sorter sorts each electromagnetic wave based on wave lengths characteristic of certain molecules, atoms, radicals or particles, based on how their atomic weights causes them to reflect light of specific bands in the spectrum. The sorter sends them to the separation chamber. Another transducer changes the waves back into particles, characteristic of their content.

The data or separation chamber isolates any particular substance or substances in the blood that is to be tested or analyzed. The computer analyzing chamber analyzes and separates the images of the particles of the different molecules, ions and particles, matching/correlating them with data in the database in the data bank.

The computer screen displays the calculable graphics, identifying all molecules, particles, ions and sub-particles in the virtual sample, again, based on atomic weights and concentrations. The concentration of the different molecules, particles, atoms and ions can be calculated by substitution methods. This may be from known to unknown methods, using a reference standard as compiled from data indices taken from actual blood samples presently in use.

The essential and major components of the workstation include the following:

A powerful ultrasonograph/ultrasonography device (may use x-rays, for example);

An ultrahigh intensity light source;

A powerful, high intensity magnetic source;

A high resolution photographic/videographic device;

An ultramicroscope, working on the principle of the electron microscope (if need be);

A high voltage, low amperage electrical source;

Crystals of appropriate sizes;

A transducer to change the images to waves, as necessary;

A cooling device to reduce the chances of damage to the tissues, due to possible overheating effects of the high intensity light source, and supersonic (ultrasound) waves;

A spectrometer/microspectrophotometry device;

A laser Doppler velocimeter;

A housing;

A second set of transducers;

A computerized data bank, with as many items in the blood recorded/coded onto a database; and

A computerized/graphic feature for analysis and calculation readout.

How the workstation 20 works will now be discussed. No contrast media will be used. There will be no invasion of the closed circulatory system. Therefore, the high intensity light source and the ultrasonography equipment producing the ultrasound, and or the ultramicroscopic device, and the magnification device, or all four combined, will be sufficiently strong to illuminate and visualize minute particles in the blood.

Ultrasosography is based on a change in the frequency of waves, as of sound or light, when the source and observer are in motion relative to each other. As readily understood by one skilled in the art, the frequency increases as the source and observer approach each other, and decrease as they move apart. In terms of the workstation 20, there may be a difference in the velocity of the blood particles in the vein (in-situ), relative to the velocity of the ‘images’ of the same particles.

Thus, v₁/v₂=x or v₂−v₁=x, where x may be a very small value. Seeing these ‘images’ may mirror wave properties associate with electrons in motion (see Busen & Kirchoff, 1860, and Sir Wm. Crookes, 1861, also, Davisson & Germer, 1927, diffracting a beam of electrons by means of a crystal lattice in a manner similar to the diffraction of x-rays, where 1/v₁−1/v₂ wavelength of each element)

Electrons in motion also have wave properties associated with it that could be described by the equation λ=h/mv, where λ=wavelength of the associated wave property, v=velocity of the electrons and m=mass. (DeBroglie, 1924).

Referencing Davisson & Germer (1927), a beam of electrons may be detracted by means of a crystal lattice, similar to diffracting x-rays. Note 1/v₁−1/v₂=λ of each element. The law of selective reflection or selective absorption by materials (Bunsen & Kirchoff, 1860 and Sir Wm. Crookes, 1861) is based on λ₁/λ₂=x or V₁/V₂=x.

The high resolution photographic and videographic devices, combined with the ultrasonography devices, will capture the images/sonograms of the molecules, atoms, ions, and particles in motion in the blood.

The magnifier is to magnify images/sonograms of particles, atoms, ions, or molecules to enable easier recognition and resolution, as they are illuminated by the ultramicroscope, in order to view tiny, sub-microscopic particles in the blood. A powerful beam of light is brought to a focus within the liquid portion of the blood, either perpendicular or at a right angle, or both, to the beam of light from the high intensity light source.

The high intensity light source is to be combined with the ultramicroscope, magnetic field and ultrasonograph.

A microspectrophotometry device, which uses the ultraviolet spectrum of the high intensity light source, is to provide a histochemical analysis—quantitative and qualitative—of the liquid portion of the blood.

A cooling system (such as liquid nitrogen, which boils at −195 degrees Celsius) is to keep the tissues cool, which are exposed to the high intensity light source and (supersonic) ultrasound waves.

The high voltage—low amperage electrical source, plus the high intensity magnetic field together, or, separate, will cause the molecules, atoms, ions, and particles, in-situ, to emit different rays/waves/beams of light, based on their respective atomic weights.

The magnetic and electric fields can, together or separately, cause the different rays/waves produced by the different molecules, atoms, ions, and particles, to fall into groups or series, and move in different paths, which are distinguishable, based on atomic weights.

A transducer will be used to convert the particulate images/sonograms to electromagnetic waves. These waves are carried to the magnetic expansion/separation chamber, where they are clustered based on wavelengths, which goes back to atomic weights.

The interphase, refractive surfaces, will refract the ultrasonic waves/beams/sonograms to provide a three dimensional view, enabling the capture of the images or sonograms of particles in the blood.

The crystals will be used to deflect the rays/waves/images/sonograms produced by each molecule, atom, ion, or particle, along different paths, to determine the wave lengths of each item in the blood.

Another transducer will change the images of waves back to particulate images, which can be isolated based on which item in the blood is ordered tested.

A computerized sorter sorts the images of particles, classifying them based on the spectra/spectrum of light reflected.

A computerized, database—data bank system receives the separated particulate images for calculation of required indices from the subject/patient/individual.

A computerized screen, on which to display analytic calculations, along with a printout of the report facility.

A summary of the key concepts will now be discussed. Beginning with the prototype, attempts will be made to make the device as compact as possible without compromising any (important) features. Miniaturization will follow very quickly, after the laboratory model is produced.

Images/sonograms of blood particles (some of which may be debris, such as broken up red blood corpuscles) molecules, atoms, ions, must be viewed, in-situ, and then transformed into virtual images/sonograms.

The use of technology to create and bridge the gap between real imaging and virtual imaging is appreciated and contemplated.

This is a technologically feasible proposition/workable device production.

The goal/objective of this device is to perform all types blood analysis, strictly, without any invasive procedures. This means that all substances in the blood and the liquid portion is to be analyzable by virtual techniques, hence the title.

Because blood is a very complex tissue—organ, containing scores of small and large particles, such as atoms, ions, molecules and fragments, in addition to the liquid on which serologic studies may also be required, the identification and analysis of any single item in the blood may not be a simple exercise.

At first glance, assumptions could be arrived at, that the images generated by ultrasound device, ultrasonography, (sonograms, 20,000 to 29,000 hertz), should be sufficient to carry out the analytic phase. But, ultrasonography provides images of gross anatomical features. When used to show the presence and certain features of a fetus, intrauterine, for example, only gross anatomical features can be shown or seen. Sometimes, even the sex of the fetus cannot be identified, with certainty. Therefore, this device anticipates the use of the following:

Ultrasosographic devices, combined with a high intensity light source, in conjunction with a high magnification device, combined with a photographic/videographic device, combined with an ultramicroscopic device, combined with a microspectrophotographic device, combined with a cooling device, in combination with an interposing transducer, and may include a second transducer; an electrical source, and a magnetic source, and a separating/sorting chamber; a computerized identification, analytic and read/print out database/data bank; a computerized animation device.

A cooling device is anticipated as being necessary because it is known that ultrasonography above 29,000 cycles per second tends to generate heat. The high intensity light source also generates heat. Since tissues are delicate and easily damaged by heat, a cooling device may be incorporated into the overall device.

Again, because of the multiplicity of images refracted/reflected by the ultrasound device and captured by the photographic/videographic devices, identification and isolation of a single item in the blood for analysis/testing may be difficult. Therefore, the use of a transducer may be needed to change the particulate images into waves. These waves will be of varying lengths, which may then be directed under the influence of either magnetic or electrical fiends, or both, to a sorting chamber. At this point in the process, we may need some form of computerized animation device to treat images as real, because, it is the picture of the images we are dealing with, not the real images.

Even before we get here, we may need the sorting chamber/device, containing crystals or prisms, or both, to sort out the waves emitted by the different substances in the blood, based on atomic weights, and the spectrum of light reflected by each element, creating a “clustering of particular waves images”. Here, if we cannot perform the required analysis, then, another transducer may be needed, to return the waves back to particulate images.

The computerized data bank will have been “programmed” with information/indices gathered from blood taken the conventional way. This database will enable us to analyze the images of whatever item in the blood we need to identify and quantify, giving the required index/indices.

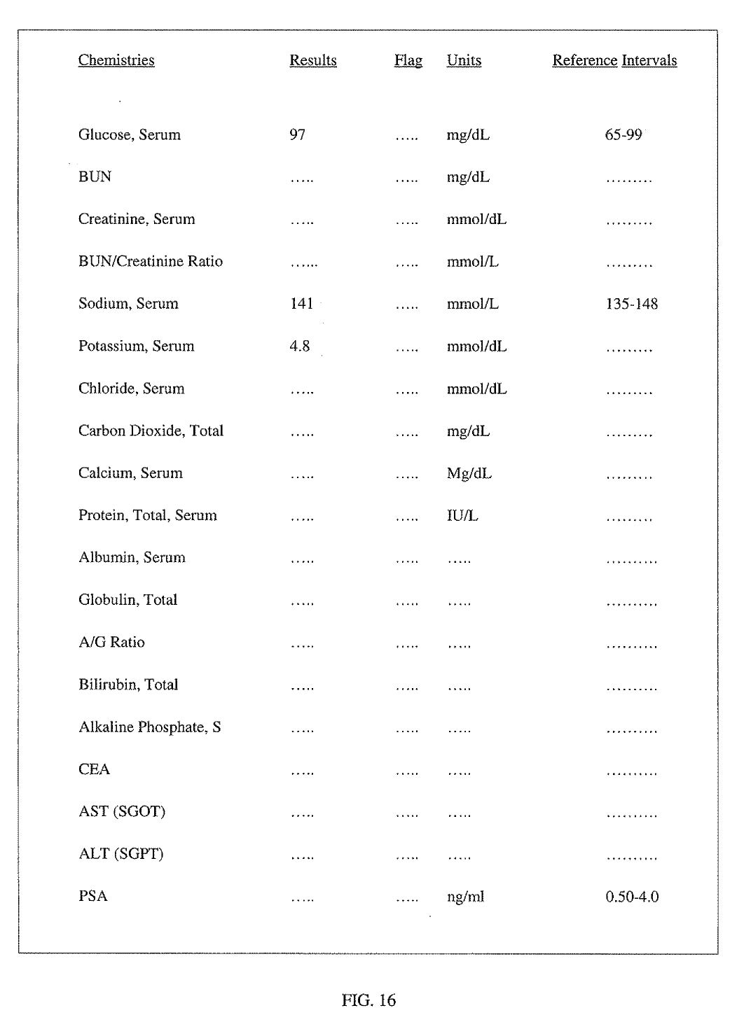

A typical laboratory order and analysis result sheet may look like the one provided in FIG. 16, which will be programmed into the data bank in a database.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. A virtual non-invasive blood analysis device workstation comprising:
a support platform for supporting a body part of a person, the body part including at least one blood vessel carrying blood;
a light source adjacent the body part for illuminating a portion of the at least one blood vessel;
a magnification device for magnifying particles of substances in the illuminated portion of the at least one blood vessel;
an imaging device for capturing images of the magnified particles of substances in the illuminated portion of the at least one blood vessel;
a transducer device for generating electromagnetic waves based on the captured images being exposed to an electromagnetic field, with the electromagnetic waves forming a plurality of color bands, with each color band corresponding to a respective particle of substance within the at least one blood vessel;
a separation chamber for separating at least a portion of the color bands within the electromagnetic waves, where at least one of the separated color bands represents current characteristics of a selected particle of substance within the at least one blood vessel;
a database of color bands representing known characteristics of the particles of substances within the at least one blood vessel; and
a processor for matching the at least one separated color band according to the selected particle of substance with at least one of the color bands in the database, and comparing the current characteristics of the at least one separated color band to the known characteristics of the at least one matched color band.

2. The virtual non-invasive blood analysis device workstation according to claim 1, further comprising an ultrasound device for generating an ultrasound image of the particles of substances in the at least one blood vessel, and providing the generated ultrasound image to said transducer.

3. The virtual non-invasive blood analysis device workstation according to claim 1, further comprising an x-ray device for generating an x-ray image of the particles of substances in the at least one blood vessel, and providing the generated x-ray image to said transducer.

4. The virtual non-invasive blood analysis device workstation according to claim 1, further comprising a display for displaying the captured images of the magnified particles of substances in the illuminated portion of the at least one blood vessel.

5. The virtual non-invasive blood analysis device workstation according to claim 1, further comprising an expansion chamber between said transducer and said separation chamber for expanding the plurality of color bands.

6. The virtual non-invasive blood analysis device workstation according to claim 1, wherein said separation chamber comprises a refracting device.

7. The virtual non-invasive blood analysis device workstation according to claim 1, wherein said imaging device comprises at least one of a still camera and a video camera.

8. The virtual non-invasive blood analysis device workstation according to claim 1, further comprising:
a temperature sensor for monitoring a temperature of the body part being illuminated by said light source; and
a cooling device for cooling the illuminated body part based on the monitored temperature.

9. The virtual non-invasive blood analysis device workstation according to claim 1, wherein at least a portion of said light source, said magnification device and said imaging device are configured as a cuff for receiving the body part.

10. The virtual non-invasive blood analysis device workstation according to claim 1, wherein at least a portion of said light source, said magnification device and said imaging device are configured as a pair of spaced apart plates for receiving the body part.

11. The virtual non-invasive blood analysis device workstation according to claim 1, wherein said support platform reflects light from said light source onto the body part of the person.

12. The virtual non-invasive blood analysis device workstation according to claim 1, further comprising at least one of a spectrometer and a spectroscope adjacent the illuminated body part.

13. The virtual non-invasive blood analysis device workstation according to claim 1, further comprising:
a second transducer for converting the electromagnetic waves after separation back to images; and
a second display for displaying the images.

14. A method for analyzing blood using a virtual non-invasive blood analysis device workstation, the method comprising:
supporting a body part of a person, the body part including at least one blood vessel carrying blood;
illuminating a portion of the at least one blood vessel using a light source adjacent the body part;
magnifying particles of substances in the illuminated portion of the at least one blood vessel using a magnification device;
capturing images of the magnified particles of substances in the illuminated portion of the at least one blood vessel using an imaging device;
generating electromagnetic waves using a transducer device based on the captured images being exposed to an electromagnetic field, with the electromagnetic waves forming a plurality of color bands, with each color band corresponding to a respective particle of substance within the at least one blood vessel;
separating at least a portion of the color bands within the electromagnetic waves using a separation chamber, where at least one of the separated color bands represents current characteristics of a selected particle of substance within the at least one blood vessel;
providing a database of color bands representing known characteristics of the particles of substance within the at least one blood vessel; and
operating a processor for matching the at least one separated color band according to the selected particle of substance with one of the color bands in the database, and comparing the current characteristics of the at least one separated color band to the known characteristics of the at least one matched color band.

15. The method according to claim 14, further comprising generating an ultrasound image of the particles of substances in the at least one blood vessel using an ultrasound device, and providing the generated ultrasound image to the transducer.

16. The method according to claim 14, further comprising generating an x-ray image of the particles of substances in the at least one blood vessel using an x-ray device, and providing the generated x-ray image to the transducer.

17. The method according to claim 14, further comprising displaying on a display the captured images of the magnified particles of substances in the illuminated portion of the at least one blood vessel.

18. The method according to claim 14, further comprising expanding the plurality of color bands using an expansion chamber between the transducer and the separation chamber.

19. The method according to claim 14, wherein the separation chamber comprises a refracting device.

20. The method according to claim 14, further comprising:
monitoring a temperature of the body part being illuminated by the light source using a temperature sensor; and
cooling the illuminated body part based on the monitored temperature using a cooling device.

21. The method according to claim 14, wherein the support platform reflects light from the light source onto the body part of the person.
